(19) United States
(12) Patent Application Publication
(10) Pub. No.: US 2006/0270959 A1
(43) Pub. Date: Nov. 30, 2006

(54) Easy open packaging device for adhesive bandage

(76) Inventors: David L. Parker, Van Nuys, CA (US); Sandra L. Parker, Van Nuys, CA (US)

Correspondence Address: Wilson Daniel Swayze, Jr., 3804 Clearwater Ct., Plano, TX, 75025, US

(21) Appl. No.: 11/196,147

(22) Filed: Aug. 3, 2005

Related U.S. Application Data
(60) Provisional application No. 60/685,597, filed on May 31, 2005.

(51) Int. Cl. A61F 15/00; A61F 13/00

(52) U.S. Cl. 602/57

(57) ABSTRACT

A package (200, 201) includes a pair of finger tabs (210, 212) formed on sheets (202, 204) that are used to enclose a sterile bandage (206) and that can be easily gripped by fingers and then pulled apart to expose the sterile bandage (206). Each finger tab (210, 212) and sheet (202, 204) is substantially identical in size and shape to provide uniformity and ease of manufacture. Finger tabs (210, 212) are positioned off-center at the end of sheets (202, 204) so that a complementary finger tab and sheet can be positioned facedown with the single finger tab positioned adjacent to the complementary finger tab, yet the finger tab is exposed without interference of the complementary finger tab to provide for ease of opening of the package. Both finger tabs may be located at the same end of the package (200, 201).

### PRIORITY

The present invention claims priority under 35 USC section 119 based on a provisional application which was filed on May 31, 2005 and has a Ser. No. 60/685,597.

### FIELD OF THE INVENTION

The present invention relates generally to packaging and dispensing elements such as a bandage package and the like and more particularly to an easily openable package for a bandage.

### BACKGROUND OF THE INVENTION

Wound dressings are commonly supplied inside a package separate from the dressing, the package serving primarily to maintain sterility of the dressing prior to application on the skin wound. With such dressings, the adhesive side of the dressing is protected by a liner which is removed at the time of application. One such package is shown in U.S. Pat. No. 4,781,293. Previous attempts have been made to provide a more efficient dressing system by including the package as part of a system. U.S. Pat. No. 4,182,449 discloses an adhesive bandage and package in which the package serves as a means of applying the bandage to the wound. In this system, the adhesive bandage includes a backing layer with a pressure sensitive adhesive layer and central pad on one surface which is partially covered with a first cover sheet such that the pad is covered and one side of the adhesive surface is releasably attached to the sheet. The partially covered bandage is placed between a pair of second cover sheets slightly larger than the bandage which are releasably sealed around the periphery to provide a sealed package with peel tabs at the end of the bandage adjacent to the partially covered portion of the bandage. To use the bandage, the peel tabs are first pulled back to beyond the partially covered portion. The first cover sheet is then lifted from the package at its end adjacent to the pad and the first cover sheet and a portion of the package on the side of the cover sheet acts as a means for applying the bandage to a skin area. This type of package for the bandage has a disadvantage in that the package is difficult to open because of the complexity involved.

### SUMMARY OF THE INVENTION

The package of the present invention includes a finger tab formed on a sheet that is used to cover the bandage and that can be easily gripped by fingers and then pulled apart to expose a sterile bandage. Each finger tab and sheet is substantially identical in size and shape to provide uniformity and ease of manufacture. The finger tab may be positioned off-center from the sheet so that a complementary finger tab and sheet can be positioned facedown so that a single finger tab is positioned adjacent to the complementary finger tab, yet the finger tab is exposed without interference of the complementary finger tab to provide for ease of opening of the package. Both finger tabs may be located at the same end of the package.

### BRIEF DESCRIPTION OF THE DRAWINGS

FIG. 1 is a perspective view of the bandage package partially open;

FIG. 2 is a top view of the unopened package;

FIG. 3 is the side view of the lower and upper sheets of the package;

FIG. 4 is the top view of an alternative unopened package;

FIG. 5 is a perspective view of a substantially square package; and

FIG. 6 is a side view of a package of one-piece construction.

### DETAILED DESCRIPTION

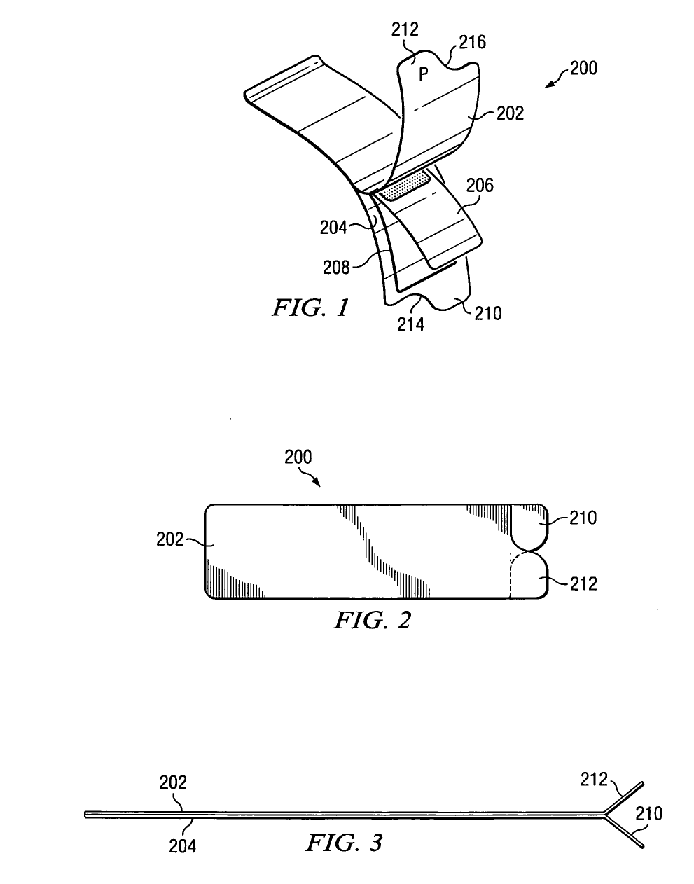

FIG. 1 shows an individual package 200 for enclosing a sterile element such as an adhesive bandage 206 which has been sealed between two facing sheets 202, 204 which are slightly larger than the bandage 206. Each facing sheet 202, 204 is substantially identical in size and shape as shown in FIG. 1 with a finger tab 212 at one end of the facing sheet 202 that is positioned off-center with respect to the longitudinal center axis of the facing sheet 202 and is to one side of one end of the facing sheet 202. The package 200 for the bandage 206 as shown in FIG. 1 is assembled with finger tabs 210, 212 at the same end of each facing sheet. Since the facing sheets 202, 204 are substantially identical, the finger tabs 210, 212 are located at the same end of each facing sheet by having one facing sheet 202 facing up and the other facing sheet 204 facing down, and each facing sheet being releasable sealed to the other. Alternatively, the substantially same structure for the package 200 could be achieved by forming a complementary facing sheet with the finger tab positioned at the same end of the facing sheet but having the finger tab located on the other side of the longitudinal center axis of the facing sheet. As shown in FIG. 6, another alternative would be to replace the two facing sheets with a single sheet being twice the length and having the finger tabs at opposite ends of the single sheet. The finger tabs could be positioned off-center to oppose each other so that when half of the sheet is folded over onto the other half, the two tabs are at the same end but are positioned side by side. The bandage 206 is enclosed within the folded sheet and sealed therein to ensure that the bandage remains sterile with a line of the releasable adhesive applied to the inner perimeter of the folded sheets. The sealing of the facing sheets may be achieved by applying a small amount of releasable adhesive which could be a line of adhesive applied to the outer perimeter of the inner surface of one or both facing sheets to maintain the bandage in a sterile condition. The finger tabs 210,212 are positioned outside of or external to the line 208 of adhesive so that the finger tabs 210,212 are free to be grasped at by the user. The facing sheets 202,204 could be constructed of different colors to provide a contrast between the facing sheets 202,204, aiding in identifying the function of the finger tabs 210,212. The user grasps both of the finger tabs 210,212 and pulls the finger tabs 210,212 apart which separates the facing sheets 202,204 exposing the inner bandage 206, eliminating the need to search for a tiny flaps or strings which are difficult to grasp even when they can be located.

FIG. 1 illustrates a perspective view of the package 200 for the bandage 206 including an upper facing sheet 202 of the package 200 and a lower facing sheet 204 of the package 200. The bandage 206 is positioned between the upper facing sheet 202 and the lower facing sheet 204 to preserve the sterile of the bandage 206. The bandage 206 may be formed of a backing layer which may be any thin, flexible sheet material suitable for covering a skin wound. The backing layer may be transparent or translucent plastic film which may be resistance to water and breathable for example impermeable to liquids and microbiological contamination but permeable to water vapor and oxygen. The backing layer may include an adhesive for attaching to the skin having similar transparency and permeability characteristics. Attached to the backing layer may be a dressing to be placed over the wound.

The package 200 as discussed above is constructed of material suitable for enclosing and protecting the bandage 206 including paper, wax paper or other materials commonly used to enclose the bandage 206 today. The upper facing 202 and the lower facing 204 of the package 200 seals the bandage 206 with a line of the adhesive 208 which is applied to the inner parameter of one or both of the facing sheets 202, 204 of the package 200. FIG. 1 additionally shows that both the upper facing sheet 202 and the lower facing sheet 204 includes a finger tab 212 and finger tab 210 respectively having a width which is approximately one half the width of the facing sheets 202,204. The finger tab 212 includes a radius 216 that allows the finger tab 212 to control the entire width of upper facing sheet 202. A similar finger tab 210 with a radius 214 on the lower facing sheet 204 has approximately the same dimensions and serves the same function for the lower facing sheet 204. Other shapes and widths for the finger tabs 210 and 212 are within the scope of the present invention. Lower facing sheet 204 is substantially identical to upper facing sheet 202 but positioned face down so that the finger tab 210 is adjacent to and side-by-side and on the same end of package 200 when the package is unopened. This arrangement of finger tab 210 and finger tab 212 allows the package 200 to be opened by grasping each finger tab 210, 212 and peeling the upper facing sheet 202 from the lower facing sheet 204, exposing and allowing the bandage 206 to be removed from the package 200. Either side of the upper facing sheet 202 and/or the lower facing sheet 204 may include information to be read either labeled or printed with instructions shown as element P in FIG. 1 such as push, pull, hold or other suitable indicia.

FIG. 2 illustrates a top view of the package 200 being unopened, and the finger tabs 210, 212 are positioned side-by-side at one end of the package 200. Although not necessary, each of the finger tabs 210, 212 could be a different color.

The upper facing sheet 202 and lower facing sheet 204 including the finger tabs 210, 212 could be formed by die cutting, laser cutting or other means of forming the facing sheets. These methods are appropriate for forming the finger tabs 210, 212 integrally with the upper facing sheet 202 and the lower facing sheet 204.

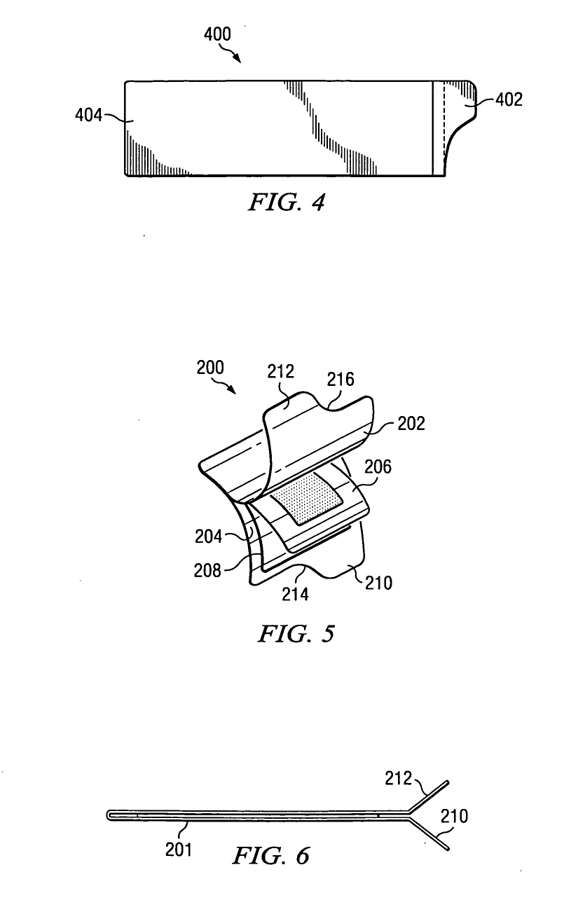

FIG. 4 shows an alternate structure and method of the invention that incorporates finger tabs 210, 212 that have been attached for example with adhesive to upper facing sheet 202 and lower facing sheet 204.

FIG. 5 illustrates a perspective view of the package 200 which is substantially square in contrast to the rectangular package of FIG. 1. Other shapes for the package 200 are within the scope of the present invention.

FIG. 6 illustrates a side view of the package 201 with a one-piece construction as discussed above. Instead of using two facing sheets to form the package 200, a single sheet having approximately twice the width or twice the length of the facing sheet shown in FIG. 1 is used to form the package 201. A single sheet which has a dimension of approximately twice the width includes a fold line along the longitudinal direction approximately in the center of the single sheet 201. The single sheet which has a dimension of approximately twice the length has a fold line in the center of the single sheet transverse to the longitudinal direction. After assembly, the package 201 should appear as seen in FIG. 2 when viewed from above.
Although embodiments of the invention have been described in the foregoing detailed description and illustrated in the accompanying drawings, it will be understood that the invention is not limited to the embodiments disclosed, and particularly to bandage applications, but is capable of rearrangements, modifications, and substitution of parts and elements as well as use in numerous devices requiring a package. The present invention is therefore intended to encompass such rearrangements, modifications and substitutions of parts and elements as fall within the spirit and scope of the invention.

1) An apparatus including a sterile element, comprising:
a first facing sheet including a first finger tab;
a second facing sheet including a second finger tab;
wherein said first facing sheet and said second facing sheet are substantially identical.

2) An apparatus including a sterile element as in claim 1, wherein said first finger tab includes a radius.

3) An apparatus including a sterile element as in claim 1, wherein said first facing sheet includes an adhesive line to attach to said second facing sheet, and wherein said first finger tab is external to said adhesive line.

4) An apparatus including a sterile element as in claim 1, wherein said first finger tab and said second finger tab lay adjacent to each other.

5) An apparatus including a sterile element as in claim 1, wherein said sterile element is a bandage.

6) An apparatus including a sterile element as in claim 1, wherein said first finger tab is a different color than said second finger tab.

7) An apparatus including a sterile element as in claim 1, wherein said first finger tab is positioned off center of said first facing sheet.

8) An apparatus including a sterile element as in claim 1, wherein said second facing sheet is face down with respect to said first facing sheet.

9) A method for forming an apparatus including a sterile element, comprising the steps of:
forming a first facing sheet including a first finger tab;
forming a second facing sheet including a second finger tab;
wherein said first facing sheet and said second facing sheet are formed to be substantially identical.

10) A method for forming an apparatus including a sterile element as in claim 9, wherein said first finger tab is formed with a radius that allows the first finger tab to control the width of said first facing sheet.

11) A method for forming an apparatus including a sterile element as in claim 9, wherein said step of forming said first facing sheet includes the step of forming an adhesive line to attach to said second facing sheet, and wherein said first finger tab is formed external to said adhesive line.

12) A method for forming an apparatus including a sterile element as in claim 9, wherein said first finger tab and said second finger tab formed adjacent to each other.

13) A method for forming an apparatus including a sterile element as in claim 9, wherein said sterile element is a bandage.

14) A method for forming an apparatus including a sterile element as in claim 9, wherein said first finger tab is formed as a different color than said second finger tab.

15) A method for forming an apparatus including a sterile element as in claim 9, wherein said first finger tab is formed off center of said first facing sheet.

16) A method for forming an apparatus including a sterile element as in claim 9, wherein said second facing sheet is formed face down with respect to said first facing sheet.

17) A method for forming an apparatus including a sterile element as in claim 9, wherein one of said first facing sheet or said second facing sheet includes material from the group consisting of paper or wax paper.

18) An apparatus including a sterile element as in claim 1, wherein said first finger tab includes information to be read.

19) An apparatus including a sterile element as in claim 1, wherein said second finger tab includes information to be read.
